(19) United States
(12) Patent Application Publication
(10) Pub. No.: US 2007/0110688 A1
(43) Pub. Date: May 17, 2007

(54) Method of dermal protection

(76) Inventor: Jonathan Roy Heylings, Syngenta (GB)

Correspondence Address: SYNGENTA CROP PROTECTION , INC.;PATENT AND TRADEMARK DEPARTMENT, 410 SWING ROAD, GREENSBORO, NC, 27409, US

(21) Appl. No.: 10/541,273

(22) PCT Filed: Jan. 12, 2004
(86) PCT No.: PCT/GB04/00091

§ 371 Date: Jan. 30, 2007

(30) Foreign Application Priority Data
Jan. 20, 2003 (GB) 0301279.6

(51) Int. Cl. A61K 8/00

(52) U.S. Cl. 424/70.1

(57) ABSTRACT

A method of dermal protection following contact between the skin and a composition containing a bipyridylium herbicide such as paraquat or diquat comprises incorporating an alginate in said composition.

This invention relates to a method of dermal protection and•in particular to a method of dermal protection following contact between the skin and a composition containing a bipyridylium herbicide.

The term “dermal protection” as used herein means a reduction of the adverse consequences of contact between the skin and a composition containing a bipyridylium herbicide. Such adverse consequences include, but are not limited to, skin irritation and acute dermal toxicity. Improved dermal protection may result from a reduction of dermal penetration of the bipyridylium herbicide or otherwise but in general a reduction of dermal penetration is indicative of improved dermal protection. It is to be understood however that the present invention results in an improved dermal protection with respect to bipyridylium herbicide formulations, including potential skin irritants conventionally contained in such formulations, and is not dependent on the mechanism by which such protection is actually achieved. Adverse skin irritation and acute dermal toxicity reactions generally arise from contact, and in particular prolonged contact, with the herbicidal concentrate prior to dilution as opposed to the herbicidal spray after dilution.

Bipyridylium herbicides have been registered for agricultural use for very many years and may be used safely and effectively if the manufacturers label recommendations are followed. Suitable precautions against accidental contact with the skin are recommended. Regulatory authorities assess the potential hazard arising from skin contact and categorize the composition accordingly. Skin irritation is defined in publicly available regulatory protocols currently in force in terms of the effects of exposure of skin to the agrochemical concentrate for a defined period, usually 4 hours. Following decontamination of the site of exposure and observation over a subsequent period, skin irritation is classified according to the regulatory criteria of National or International Regulatory Authorities such as the EU. Alternative methods for assessing skin irritation are being developed for regulatory purposes and otherwise and are also available the determination skin irritation potential. Dermal Toxicity is defined by the dose of the formulation (mg/kg) that evokes a systemic toxic response via the dermal route. Clearly any reduction in skin irritation or dermal toxicity is highly desirable.

In EP 0467529 there is described a liquid aqueous herbicidal composition comprising a salt of paraquat or diquat or a mixture thereof, in a concentration of at least 50 grams per litre, in admixture with a suspension of from 10 to 400 grams per litre of a magnesium trisilicate, the composition further comprising an emetic and/or purgative. The magnesium trisilicate forms a gel at the pH of the human gastric juice and the specification further discloses an aqueous liquid herbicidal comprising: (i) a herbicidal component comprising a salt of paraquat or diquat, or a mixture thereof; (ii) a gelling agent that will gel at the pH of human gastric juice; and (iii) an emetic and/or a purgative; wherein the ratio of the herbicidal component to the gelling agent is from 1:1 to 20:1. The object of the invention is to reduce the possibility of harmful effects following the ingestion of a bipyridylium salt. Thus if a quantity of a composition according to the invention is ingested, the acidity of the gastric juice (which varies within quite wide limits but has a mean value of about pH 1.92 for men and pH 2.59 for women) will cause the composition to gel in the stomach. Increasing the viscosity of the gastric contents slows down the rate of gastric emptying. The bipyridylium herbicide will consequently be trapped in the gel, and its movement from the stomach and into the absorptive small intestine will be impeded. The emetic present in the composition is absorbed relatively rapidly and will in a short time cause expulsion of the gel containing the bipyridylium herbicide by vomiting, thereby preventing the ingested herbicide from moving further down the gastrointestinal tract, where absorption of the bipyridylium compound would otherwise take place. In preferred compositions a purgative is present in the composition, to assist in removing any non-absorbed bipyridylium herbicide that has passed from the stomach into the small intestine despite the action of the emetic. In the event of a bipyridylium composition according to the invention of EP 0467259 being ingested, the combined effects of the gelling agent, emetic, and when included, the purgative, will substantially reduce the absorption of the bipyridylium compound from the gastrointestinal tract into the bloodstream, and thereby to reduce the oral toxicity of the product.

The formulation described in EP 0467259 proved in practice not to be commercially viable. It was found essential to include a thickening or suspending agent to assist in keeping the particles of the insoluble gelling agent, magnesium trisilicate, evenly dispersed throughout the composition during storage and transport. However by its very nature the thickening agent increased the viscosity of the composition and a balance had to be struck between the problems associated with a high-viscosity composition and the need to increase viscosity to minimise settling of the solid inorganic gelling agent. In practice the balance proved an unhappy compromise in that the composition had relatively poor stability as regards settling of the solid gelling agent yet still proved excessively viscous resulting in difficulty in pouring and measuring the composition, difficulty in dispersing the composition effectively in water in the spray tank and difficulty in rinsing empty containers. Settling of the dispersed solid inorganic gelling agent may lead to a concentration gradient of magnesium trisilicate versus emetic such that if only a proportion of a container of formulation is used at any one time, the relative proportions of the ingredients present in the spray tank will not correspond to those intended and the safening effect may in consequence be far from than optimum. The preferred thickening or suspending agent is the xanthan gum sold under the tradename KELZAN and this is the sole suspending agent used in the examples. There is however a brief comment that other suitable suspending agents include alginates.

In WO 02/076212 it is disclosed that that alginates themselves are surprisingly effective pH-sensitive gelling agents for use with bipyridylium salt formulations when used as the pH-sensitive gelling agent. WO 02/076212 therefore discloses the use of an alginate as a pH-triggered gelling agent in the manufacture of a composition comprising a salt of paraquat, a salt of diquat or a mixture thereof, the composition further comprising an emetic and/or purgative such that a pH-triggered gel effect takes place at the acid pH of human gastric juice.

It will be understood that the inventions described in EP 0467529 and WO 02/076212 are directed entirely to the mitigation of the oral toxicity of bipyridylium herbicide concentrate formulations when deliberately or accidentally ingested. The mechanism relies on the physical ejection of the gelled composition from the stomach by vomiting before it can be absorbed and by purgation to assist in removing any non-absorbed bipyridylium herbicide which has passed from the stomach into the small intestine despite the action of the emetic. We have now found that alginates have a surprising effect in reducing the skin irritation and/or dermal toxicity following contact between the skin and a bipyridylium herbicide composition. That effect is not to be expected given the teaching of WO 02/076212 and the entirely different mechanism by which alginate acts to gel at the pH of the stomach and is then physically ejected by vomiting.

Solid calcium alginate is used, typically in the form of a non-woven sheet or a swatch of fibres or rope, as a dressing for wounds and in particular for heavily exuding chronic wounds such as venous leg ulcers, diabetic ulcers and pressure ulcers. A review is published in Wound Care newsletter, October 1998 and discloses that the alginate fibre absorbs and interacts with fluid extrudate from the wound and turns into a hydrophilic gel. Alginates tend to keep mounds moist and foster the formation of granulation tissue. Upon removal, the dressing can be washed away with saline irrigation.

According to the present invention there is provided a method of dermal protection following contact between the skin and a composition comprising a bipyridylium herbicide which method comprises incorporating an alginate in said composition.

The term bipyridylium herbicide includes paraquat, diquat and a mixture of paraquat and diquat. Paraquat and diquat are normally formulated in the form of agriculturally acceptable, water-soluble salts. The composition for use in the present invention is suitably an aqueous concentrate intended to be diluted prior to application.

Thus aqueous compositions according to the invention suitably contain at least 25 grams per litre, for example at least 40 grams per litre of paraquat or diquat or mixtures thereof (individually or in combination referred to herein as bipyridylium salt) expressed as bipyridylium ion. The compositions may contain greater than 50 grams per litre, for example greater than 100 grams per litre of bipyridylium ion. Compositions containing 200 grams or more per litre may be prepared although a concentration of paraquat in excess of about 250 or 350 g/l approaches the upper limit where composition stability becomes a problem. In general compositions do not contain greater than 400 grams per litre of bipyridylium ion. Thus a typical concentrate composition contains from 50 grams per litre to 250 grams per litre of bipyridylium ion.

The term alginate as used herein means the class of natural block copolymers extracted from seaweed and consisting of uronic acid units, specifically 1-4a, L-guluronic and 1-4b, D-mannuronic acid, connected by 1:4 glycosidic linkages. The general structure is illustrated in FIG. 1below.

[image]

[image]

The ratios of mannuronic/guluronic acid residues (M:G) vary depending on the algal source. Typically alginates are classified as being “high-G” or “high-M”. Alginates are often sold in the form of the sodium salt but different commercial grades may contain varying proportions of residual calcium ion.

The mechanism by which the alginate operates to achieve dermal protection is not understood and several alternative theories may be produced by way of explanation. It is clear however that, whatever the mechanism, it is very different from that of the “triggered gel” described in WO 02/076212. In the first place, whilst skin may in some circumstances be mildly acidic, it is much less so than the stomach. The stomach is in effect an acidic container that receives the swallowed composition and on contact with the highly acidic gastric juice, the composition gels. This aids its removals from the body by vomiting. In contrast, the skin is a neutral or very mildly acidic surface that is most unlikely to “trigger” any significant gelling action. Furthermore, when a liquid composition contacts the skin it will immediately start to dry out. Skin irritation results at least in part from the skin penetration characteristics of the bipyridyl from the residue left as the composition dries onto the skin. It is likely that the alginate has a skin protectant action as the composition dries on the skin but exactly how this is achieved is unknown.

There does not appear to be any connection between the mechanism by which a solid calcium alginate dressing assists in the healing of heavily exuding chronic wounds and the skin protectant action of the present invention.

We have found that the addition of alginates does not necessarily provide significant dermal protection against skin irritant agrochemical compositions in general and the dermal protection action of the alginate appears to be largely specific to herbicide compositions containing bipyridylium ion. The reason for this is not known.

Examples of commercially available alginates suitable for use in the method of the present invention are shown in the following Table: —

| | Monomer | | 1% Viscosity | Approx. molecular | pH of 1% |
|---|---|---|---|---|---|
| Alginate | ratio | Ca²⁺ content | (mPas) | weight | solution |
| MANUTEX RM | high M:G | low Ca²⁺, 0.4% max | 200-400 | 120,000-190,000 | 5.0-7.5 |
| MANUTEX RD | high M:G | low Ca²⁺, 0.4% max | 4-15 | 12,000-80,0000 | 5.0-7.5 |
| KELGIN HV | high M:G | high Ca²⁺, 1.5% max | 600-900 | 120,000-190,000 | 6.4-8.5 |
| KELGIN LV | high M:G | high Ca²⁺, 1.5% max | 40-80 | 80,000-120,000 | 6.4-8.5 |
| MANUGEL | high G:M | low Ca²⁺, 0.2-0.5% | 110-270 | 80,000-120,000 | 5.0-7.5 |
| GMB | | | | | |
| MANUGEL | high G:M | low Ca²⁺, 0.2-0.5% | 50-100 | 80,000-120,000 | 5.0-7.5 |
| GHB | | | | | |
| KELCOSOL | high M:G | high Ca²⁺, 1.5% max | 1000-1500 | 120,000-190,000 | 6.4-8.0 |

The dermal protection provided is not critically dependent on the molecular weight of the alginate. The average molecular weight of the alginate is preferably from 5,000 to 250,000 for example 10,000 to 250,000, and in particular from 10,000 to 200,000. We have found for example that good dermal protection is provided using both MANUTEX RM (molecular weight 120,000 to 190,000) and MANUTEX RD (molecular weight 12,000 to 80,000). Mixtures of different grades of alginate may be used if desired. The molecular weight of the alginate is reflected in the viscosity of its solution in water under a defined set of conditions. Preferred alginates have an average viscosity in a 1% aqueous solution (referred to herein as the “1% Solution Viscosity”) of from 2 to 2000 mPas, for example from 2 to 1,500 mPas and especially from 2 to 1000 mPas and preferably from 4 to 450 mPas at 25° C. as measured using an LV model of the BROOKFEELD viscometer (Brookfield Engineering laboratory, Stoughton, Mass.) at 60 rpm with a number 3 spindle.

Especially preferred alginates are those sold under the trade name MANUTEX RM and MANUTEX RD. MANUTEX, MANUGEL, KELGIN and KELCOSOL are trademarks of ISP. The concentration of alginate in the composition will generally range from 1 to 50 g/l, for example from 3 to 50 g/l or in the alternative from 5 to 20 g/l, and in particular from 5 to 15 g/l. Higher concentrations may be used if desired but may tend to increase the viscosity of the composition beyond what is acceptable in commercial practice whilst a concentration of below 3 g/l may not provide sufficient dermal protection. The pH of the composition is not critical in terms of the dermal protection provided by the alginate, and pH of the bipyridyl composition may be used at its natural pH or may be adjusted to if desired to improve stability or for any other reason. For paraquat compositions typical values are between about pH 4 and pH 9 for example between about pH 6.5 and pH 7.5 and in particular about pH 7. For diquat the pH is more usually adjusted to between about pH 5 to pH 6. Conventional acids or bases such as acetic acid or sodium hydroxide may be used if desired to adjust the pH of the composition.

A high viscosity of the formulation at its natural pH is undesirable for most applications and it is preferred that the viscosity of the formulation of the invention (“composition viscosity”) as measured using the method of Example 1 is below 300 mPas, and preferably below 200 mPas for example from 10 to 250 mPas and preferably from 20 to 200 mPas. It will be recognised however that a high viscosity formulation, for example having a viscosity of 300 mPas or more, may have utility in some specialised applications. The viscosity of the composition will of course depend on the totality of its content including any surfactants present

The scope of the present invention is not restricted to any specific aqueous bipyridylium herbicide composition. It is clearly desirable however that the alginate should provide dermal protection in respect of commercially useful concentrates which frequently contain conventional adjuvants or other additives. Commercial compositions generally include one or more surfactants or adjuvants in the composition to improve the bioperformance of the herbicide. Such surfactants are well known to those skilled in the art and include cationic, non-ionic and anionic compounds. Examples are listed in EP 0467529 the disclosure of which is incorporated by reference. When one or more surfactants is present the total surfactant concentration is preferably from 25 to 200 g/l of the composition, preferably from 50 to 150 g/l for example from 50 to 100 g/l and in particular 50 to 70 g/l. It is to be understood that the presence of alginate increases the dermal protection in respect of the composition taken as a whole, including the bipyridylium herbicide, surfactants and other components that may be present as described below. Surfactants included to enhance biological performance may contribute to adverse dermatological effects. In general however we have found that the action of the alginate is associated primarily with the bipyridylium herbicide composition and that dermal protection is not necessarily provided in respect of aqueous compositions containing only surfactant. Indeed a bipyridylium composition containing skin irritant surfactants may show a greater skin irritation even in the presence of alginate than a corresponding composition containing alginate but no surfactant. What is important however is that a bipyridylium composition containing alginate and surfactant shows a lower skin irritation than the corresponding composition containing surfactant but no alginate.

Examples of typical anionic surfactants include a salt of an alkyl benzene sulfonate such as sodium or magnesium dodecyl benzene sulfonate (commercially available examples include NANSA HS90/S); alkyl ethoxy carboxylates, for example those of general formula R(OCH₂CH₂)ₙOCH₂CO₂H. where R=C₁₂-C₁₄ alkyl and n=6 to 12 (commercially available examples include EMPICOL CBF and EMPICOL CBL);

disodium C₅ to C₂₀ straight or branched chain alkyl sulfosuccinates such as disodium lauryl sulfosuccinate and disodium isodecyl sulfosuccinate (commercially available examples include AEROSOL A268); sodium di(C₅ to C₁₂ straight or branched chain) alkyl sulfosuccinates such as sodium dioctyl sulfosuccinate (commercially available examples include AEROSOL OT); sodium alkyl sulfosuccinates such as sodium lauryl sulfosuccinate (commercially available examples include TEXIN 128 P); sodium naphthalene formaldehyde condensates (commercially available examples include MORWET D425); sodium methyl oleoyl taurate (commercially available examples include ADINOL OT64); ester carboxylates (commercially available examples include EURACOL M, TA); phosphate esters (commercially available examples include CRODAFOS); TEA-PEG-3 cocamide sulfate (commercially available examples include GENAPOL AMS).

Examples of typical non-ionic surfactants include nonyl phenol ethoxylates (commercially available examples include SYNPERONIC NP8); block copolymers of ethylene oxide and propylene oxide (commercially available examples include SYNPERONIC PE/F88); alkyl amine ethoxylate (commercially available examples include SYNPROLAM 35×15, ETHOMEEN C25 or T25 and NOVAMINE); ethoxylated linear alcohols (commercially available examples include LUBROL 17A17; other alcohol ethoxylates (commercially available examples include SYNPERONIC A range (11, 15, 20, etc), ATPLUS 245); and fatty acid ethoxylates (commercially available examples include CHEMAX). It may be noted that surfactants such as alkylamine ethoxylates are sometimes classified as cationic surfactants, but at neutral pH as in most compositions of the present invention they are properly considered to be non-ionic.

Examples of typical cationic surfactants include amine ethoxylates and alkoxylated diamines (commercially available examples include JEFFAMINE products).

Paraquat is the common name of the 1,1′-dimethyl-4,4′-bipyridylium cation. Diquat is the common name of the 1,1′-ethylene-2,2′-bipyridylium cation. Salts of paraquat and diquat necessarily contain anions carrying sufficient negative charges to balance the two positive charges on the bipyridylium nucleus.

Since the characteristic herbicidal effect of a bipyridylium quaternary cation is independent of the nature of the associated anion, the choice of the anion is a matter of convenience, depending, for example, on cost. Preferably the anion is one which gives rise to a salt of convenient water solubility. Examples of anions, which may be mono- or polyvalent, include acetate, benzenesulfonate, benzoate, bromide, butyrate, chloride, citrate, fluorosilicate, fumarate, fluoroborate, iodide, lactate, malate, maleate, methylsulphate, nitrate, propionate, phosphate, salicylate, succinate, sulphate, thiocyanate, tartrate, and p-toluenesulfonate. The salt of the herbicidal bipyridylium cation may be formed from a number of similar anions or mixtures of different ones. For reasons of convenience and economy, paraquat is normally manufactured and sold as paraquat dichloride while diquat is manufactured and sold as diquat dibromide.

Since the characteristic herbicidal activity of a salt of a herbicidal bipyridylium quaternary cation resides in the cation only, it is customary to quote concentrations of active ingredient and rates of application in terms of the amount of bipyridylium quaternary cation unless otherwise stated.

If desired the paraquat or diquat may be used in the formulation of the present invention in combination with another agrochemical active ingredient and in particular with another herbicide. Paraquat and diquat mixtures are also useful as agrochemical active ingredient in the present invention. Typical mixture partners for paraquat and diquat useful for incorporation in compositions of the present invention include ametryn, diuron, atrazine, glyphosate, butafenacil, metribuzin, prometryn, and terbutylazine. Many other possible mixture partners which may either be incorporated in a composition of the present invention or used in a tank mix with a composition of the present invention will occur to those skilled in the art. Representative examples include 2,4-D, AC304415, Acetochlor, Aclonifen, Alachlor, Amicarbazone, Aminotriazole, Azafenidin, BAS145138, Benoxacor, Bentazon, Bialophos, Bromoxynil, Butylate, Carfentrazone-ethyl, CGA 276854, Clomazone, Clopyralid, Cloquintocet-mexyl, Cloransulam, Cyanazine, Dicamba, Dichlormid, Diclosulam, Diflufenzopyr, Dimethanamid, Fenclorim, Fentrazimide, Florasulam, Flufenacet, Flumetsulam, Flumiclorac-pentyl, Flumioxazin, Flurazole, Fluroxypyr, Fluthiacet-methyl, Fluxofenim, Foramsulfuron, Furilazole, Glufosinate, Halosulfuron-methyl, Halosulfuron-methyl, Imazamox, Imazapyr, Imazaquin, Imazethapyr, Iodosulfuron, Isopropazol, Isoxachlortole, Isoxaflutole, MCPA, MCPB, MCPP, Mefenpyr, Mesotrione, Metobenzuron, Metolachlor, Metosulam, MON4660, Nicosulfuron, NOA-402989, Pendimethalin, Primisulfuron, Profluazol, Prosulfuron, Pyridate, Rimsulfuron, S-Dimethanamid, Sethoxydim, S-glufosinate, Simazine, Slurtamone, S-Metolachlor, Sulcotrione, Sulfentrazone, Sulfosate, Terbutryn, Thifensulfuron and Tritosulfuron.

It will be appreciated that, whilst the novel method of the present invention concerns improved dermal protection, compositions for use in the method of the invention will normally also be formulated to reduce the effects of accidental or deliberate ingestion and will generally contain a conventional emetic.

A variety of known emetics may be used in the compositions for use in the method of the invention. However, preferred emetics are those compounds disclosed in UK Patent No. 1507407 for use in formulations of bipyridylium herbicides, and a particularly preferred emetic is 2-amino-6-methyl-5-oxo-4-n-propyl-4,5-dihydro-5-triazolo[1,5-a]-pyrimidine.

The amount of emetic used in the composition will vary depending upon the particular type of emetic used, but when an emetic of the class disclosed in UK Patent No. 1507407 is used, the concentration of emetic is preferably from 0.1 to 5 grams per litre of the composition. For a composition containing 200 grams per litre of bipyridylium compound, a concentration of from 1.0 to 2.0 grams per litre and in particular from 1.5 to 2.0 grams per litre of emetic is preferred.

For some applications the composition of the invention may additionally contain a purgative, for example magnesium sulphate.

The concentration of magnesium sulphate, when used, is preferably from 10 to 400 grams per litre of the composition based on the weight of dry magnesium sulphate containing no water of hydration, and more preferably from 10 to 100 grams per litre. Higher concentrations of magnesium sulphate, for example up to 400 grams per litre, may be used and may continue to provide increased purgative effect but such high levels of magnesium sulphate may have an adverse effect on formulation stability. The composition for use in the method of the invention may also contain conventional additive such as an odourant (alerting agent), for example as a pyridine derivative, as described in UK Patent No. 1406881, or n-valeric acid. The compositions may also comprise a pigment or a dye to give them a distinctive colour.

Compositions for use in the method of the present invention may be prepared simply and conveniently by mixing the components. Solid alginate may be added to an aqueous solution of the bipyridylium salt or the alginate may first be mixed into water and subsequently added to an aqueous solution of bipyridylium.

The invention is illustrated by the following Examples in which all parts and percentages are by weight unless otherwise stated. The concentration of adjuvants is in each case given in terms of the weight of composition used. The concentration of adjuvant in the composition is given when it is less than 100%. For example the product NANSA HS90/S is supplied as a 90% by weight solution of sodium dodecyl benzene sulfonate.

In Example 1, skin irritation was measured using the published Regulatory Protocol OECD 404 and 402. In subsequent Examples, skin irritancy was measured using an in vitro skin irritation function test (SIFT) based on the electrical resistance of excised mouse skin and a test based on the skin penetration of the test chemical itself in this in vitro model. The test, which reduces the amount of animal testing required, is described in “A prevaldiation study on the in vitro skin irritation function test (SIFT) for prediction of acute skin irritation in vivo: results and evaluation of ECVAM Phase III, Heylings, Diot, Esdaile, Fasano, Manning and Owen, Toxicology in Vitro 17 (2003) 123-136 which is incorporated herein by reference. The paper reports a Phase III validation of the test using various chemicals. Results of the SIFT test are reported as electrical resistance in k Ohms after 20 hours exposure to the test composition. The magnitude of any reduction in electrical resistance is indicative of the degree of skin irritation.

The SIFT protocol identifies changes in barrier function of the skin following topical exposure to potential skin irritants. It is also highly desirable to measure the penetration of the test chemical itself in this in vitro model since skin penetration increases as the barrier is damaged. The SIFT test was therefore refined to incorporate the measurement of bipyridyl chemicals e.g. paraquat. The methodology is as described above in Heylings et al, apart from the addition of radiolabelled paraquat to the dosing solution prior to application to the skin. The dosing preparation is tested for homogeneity to ensure that the radiolabel is fully dispersed into the formulation and at sufficient specific activity to allow adequate measurement in the test system. At a time point of 4 hours following topical skin exposure, a sample of the saline receptor fluid bathing the underside of the skin was taken and the amount of paraquat that has penetrated the skin at this time point was measured by liquid scintillation counting. Validation of this model with in vivo measurements has shown that the lower the amount of penetration of paraquat through the skin then the less the in vivo skin irritation response, as measured by the conventional OECD regulatory test. The performance of a composition in this in vitro SIFT protocol, in terms of the ability of a new composition to reduce the skin penetration of paraquat, is compared with its relevant control. Thus, percentage values below 100 demonstrate a potential benefit. Two additional internal positive and negative controls were included in this test that confirm that the equipment and the batch of skin are performing adequately. Standards used were the skin irritant, sodium lauryl sulphate (positive control) and untreated skin (negative control), as described in Heylings et al above. In general, we have found that compositions containing both paraquat and alginate reduced the penetration of paraquat through the skin compared to compositions that did not contain alginate. This has been substantiated in vivo where such compositions containing alginate are less irritant to the skin than those without alginate.

### EXAMPLE 1

A composition (Composition 1) for use in the method according to the present invention was prepared having the following components: —

| COMPONENT | CONCENTRATION | |
|---|---|---|
| Paraquat dichloride | 200 | g/l |
| | | (paraquat ion) |
| SYNPROLAM 35 × 15 | 31 | g/l |
| AEROSOL OT-B | 22.35 | g/l |
| MANUTEX RM | 10 | g/l |
| Magnesium sulphate containing 1.5 molecules | 74 | g/l |
| of water of hydration | | |
| Acetic Acid | To pH 6.5-7.5 | |
| Emetic | 1.5 | g/l |
| 2-amino-6-methyl-5-oxo-4-n-propyl-4,5- | | |
| dihydro-5-triazolo[1,5-a]-pyrimidine | | |
| Colourant | 2.5 | g/l |
| Antifoam | 0.25 | g/l |
| Odour | 0.1 | g/l |

| | |
|---|---|
| Water | To 1 litre |

AEROSOL OT-B contains 85% sodium dioctyl sulfosuccinate and 15% sodium benzoate. SYNPROLAM 35×15 is an alkyl amine ethoxylate with a molecular formula that can be written as R—N(CH₂CH₂O)ₓH(CH₂CH₂0)_{y}H where the sum of x and y is 15 and R=C₁₃-C₁₅. MANUTEX RM is a high M alginate having a low calcium content (0.4% maximum) and a 1% solution viscosity of 200 to 400 mPas.

The composition had a viscosity as measured using a Paar Physica Haake MCl+High Shear Rheometer at 25° C. at 300 s⁻¹ (“composition viscosity”) of 68.0 mPas.

The above composition was evaluated for skin irritation and dermal toxicity using the published Regulatory Protocol OECD 404 and 402. This showed a significant reduction in skin irritancy and dermal toxicity as compared with data on the commercial paraquat product containing no alginate.

### EXAMPLES 2-6

In these Examples alginate was added to the composition shown in Table 2.

TABLE 2

| COMPONENT | CONCENTRATION | |
|---|---|---|
| Paraquat dichloride | 100 | g/l |
| | | (paraquat ion) |
| Genamin T-150 | 15.5 | g/l |
| NANSA 1169-A | 31.6 | g/l |
| Magnesium sulphate containing 1.5 molecules | 74 | g/l |
| of water of hydration | | |
| Acetic Acid | To pH 6.5-7.5 | |
| Emetic | 1.5 | g/l |
| 2-amino-6-methyl-5-oxo-4-n-propyl-4,5- | | |
| dihydro-5-triazolo[1,5-a]-pyrimidine | | |
| Colourant | 2.5 | g/l |
| Antifoam | 0.5 | g/l |
| Odour | 10 | g/l |

| | |
|---|---|
| Water | To 1 litre |

NANSA 1169-A is a 30% w/w aqueous solution of Sodium Dodecyl Benzene Sulfonate. Genamin T-150 is an alkyl amine ethoxylate with a molecular formula that can be written as R—N(CH₂CH₂O)ₓH(CH₂CH₂0)_{y}H where the sum of x and y is 15 and R=tallow.

The actual alginate added, its concentration and the skin irritation value of the resultant composition using the SIFT test are shown in Table 3. In Table 3 the skin irritancy value is measured in terms of the percentage reduction of skin irritancy (as measured by paraquat skin penetration) relative to a relevant commercial standard containing 100 g/l paraquat and no alginate. Thus values less than 100% show a reduced skin irritancy value relative to the standard. Data are mean values from a minimum of six observations in the SIFT protocol.

TABLE 3

| Example | | | |
|---|---|---|---|
| Number | | | Relative Skin |
| All contain | | Concentration | Irritancy Value |
| 100 g/l | | of Alginate | % paraquat skin |
| paraquat | Alginate | (g/l) | penetration |
| 2 | MANUTEX RM | 7 | 85% |
| | MANUTEX RD | 7 | |
| 3 | MANUTEX RM | 5 | 76% |
| | MANUTEX RD | 5 | |
| 4 | MANUTEX RM | 3.5 | 63% |
| | MANUTEX RD | 3.5 | |
| 5 | MANUTEX RM | 7 | 47% |
| 6 | MANUTEX RD | 7 | 77% |

### EXAMPLES 7 TO 19

In Table 4 the skin irritancy value is measured in terms of the percentage reduction of skin irritancy (as measured by paraquat skin penetration) relative to a relevant pre-commercial standard containing 200 g/l paraquat and no alginate. Thus values less than 100% show a reduced skin irritancy value relative to the standard. Data are mean values from a minimum of six observations in the SIFT protocol.

TABLE 4

| | Example | | | | | | | |
|---|---|---|---|---|---|---|---|---|
| | No. | | | | | | | |
| Component | 7 | | 8 | | 9 | | 10 | |
| Paraquat dichloride | 200 | g/l | 200 | g/l | 200 | g/l | 200 | g/l |
| | | (paraquat ion) | | (paraquat ion) | | (paraquat ion) | | (paraquat ion) |
| Emetic | 0.5 | g/l | 1.5 | g/l | 1.5 | g/l | 1.5 | g/l |
| NANSA 1169A | 63.3 | g/l | 63.3 | g/l | 63.3 | g/l | 63.3 | g/l |
| GENAMIN T150 | 31 | g/l | 31 | g/l | 31 | g/l | 31 | g/l |
| MANUTEX RM | 9 | g/l | 9 | g/l | 9 | g/l | 9 | g/l |
| Magnesium Sulphate | 74 | g/l | 74 | g/l | 74 | g/l | 74 | g/l |
| 1.5 H₂O | | | | | | | | |
| Antifoam | 0.25 | g/l | 0.25 | g/l | 0.25 | g/l | 0.25 | g/l |
| Colorant | 2.5 | g/l | 2.5 | g/l | 2.5 | g/l | — | |
| Odour | 0.1 | g/l | 0.1 | g/l | 0.1 | g/l | 0.1 | g/l |

| Acetic acid | To pH 6.5-7.5 | To pH 9 | To pH 5 | To pH 6.5-7.5 |
|---|---|---|---|---|
| Water | To 1 Litre | To 1 Litre | To 1 Litre | To 1 Litre |
| Skin Irritation value | 60% | 58% | 57% | 52% |
| % paraquat skin | | | | |
| penetration | | | | |
| | Example No | | | |

| | | | |
|---|---|---|---|
| Component | 11 | 12 | 13 |

| Paraquat dichloride | 200 | g/l | 200 | g/l | 200 | g/l |
|---|---|---|---|---|---|---|
| | | (paraquat ion) | | (paraquat ion) | | (paraquat ion) |
| Emetic | 1.5 | g/l | 1.5 | g/l | 1.5 | g/l |
| NANSA 1169A | — | | 63.3 | g/l | — | |
| GENAMIN T150 | 31 | g/l | — | | — | |
| MANUTEX RM | 9 | g/l | 9 | g/l | 9 | g/l |
| Magnesium Sulphate | 74 | g/l | 74 | g/l | 74 | g/l |
| 1.5 H₂O | | | | | | |
| Antifoam | 0.25 | g/l | 0.25 | g/l | 0.25 | g/l |
| Colorant | 2.5 | g/l | 2.5 | g/l | 2.5 | g/l |
| Odour | 0.1 | g/l | 0.1 | g/l | 0.1 | g/l |

| | | | |
|---|---|---|---|
| Acetic acid | To pH 6.5-7.5 | To pH 6.5-7.5 | To pH 6.5-7.5 |
| Water | To 1 Litre | To 1 Litre | To 1 Litre |
| Relative Skin | 52% | 26% | 14% |
| Irritation value (%) | | | |
| based on paraquat | | | |
| skin penetration | | | |

### EXAMPLE 14

Example 14 further illustrates the reduction in skin irritation obtained by the addition of alginate in respect of paraquat. In this Example the Relative Skin Irritation Value is expressed as a percentage of the value obtained for the exactly corresponding composition containing no alginate.

| | Example | |
|---|---|---|
| Component | No 14 | |
| Paraquat dichloride | 200 | g/l |
| | | (paraquat ion) |
| Emetic | 0.5 | g/l |
| BioSoft SDBS 30LA | 86 | g/l |
| GENAMIN T150 | 43 | g/l |
| MANUTEX RM | 9 | g/l |
| Magnesium Sulphate heptahydrate | 123.74 | g/l |
| Antifoam | 0.5 | g/l |
| Colorant | 2.5 | g/l |
| Odour | 0.1 | g/l |

| | |
|---|---|
| Acetic acid | To pH 6.5-7.5 |
| Water | To 1 Litre |
| Relative Skin Irritation value (%) | 78% |
| based on paraquat skin penetration | |

BioSoft SDBS 30LA is a 30% w/w aqueous solution of sodium dodecyl benzene sulfonate.

1. A method of dermal protection following contact between the skin and a composition comprising a bipyridylium herbicide which method comprises incorporating an alginate in said composition.

2. A method according to claim 1 in which the bipyridium herbicide is a paraquat salt

3. A method according to claim 1 in which the bipyridylium herbicide is a diquat salt.

4. A method according to claim 1 in which the bipyridium herbicide is a mixture of a paraquat salt and a diquat salt.

5. A method according to claim 1 wherein the composition is an aqueous concentrate composition containing at least 25 grams per litre of bipyridylium ion.

6. A method according to claim 5 wherein the composition contains from 50 grams per litre to 250 grams per litre of bipyridylium ion.

7. A method according to claim 1 wherein the average molecular weight of the alginate is 5,000 to 250,000.

8. A method according to claim 7 wherein the average molecular weight is from 10,000 to 200,000.

9. A method according to claim 1 wherein the 1% Solution Viscosity of the alginate is from 2 to 2000 mPas.

10. A method according to claim 9 wherein the 1% Solution Viscosity of the alginate is from 2 to 1,500 mPas.

11. A method according to claim 1 wherein the concentration of the alginate in the composition is from 1 to 50 g/l.

12. A method according to claim 11 wherein the concentration of the alginate in the composition is from 5 to 15 g/l.

13. A method according to claim 1 wherein the composition contains one or more surfactants.

14. A method according to claim 1 wherein the composition contains an emetic.

15. A method according to claim 1 wherein the composition contains a purgative.
